(19) United States
(12) Patent Application Publication
(10) Pub. No.: US 2018/0310922 A1
(43) Pub. Date: Nov. 1, 2018

(54) METHODS AND APPARATUS FOR PERFORMING AT LEAST THREE MODES OF ULTRASOUND IMAGING USING A SINGLE ULTRASOUND TRANSDUCER

(71) Applicant: Clarius Mobile Health Corp., Burnaby (CA)

(72) Inventors: Laurent PELISSIER, North Vancouver (CA); Kris DICKIE, Vancouver (CA); Benjamin Eric KERBY, Richmond (CA); Nishant UNIYAL, Vancouver (CA); Reza Zahiri, Burnaby (CA)

(21) Appl. No.: 15/817,003

(22) Filed: Nov. 17, 2017

Related U.S. Application Data
(60) Provisional application No. 62/424,152, filed on Nov. 18, 2016.

(51) Int. Cl. A61B 8/00; A61B 8/14

(52) U.S. Cl. CPC A61B 8/54; A61B 8/145; A61B 8/4488; A61B 8/4494

(57) ABSTRACT

This disclosure relates to methods and apparatus for performing at least three modes of ultrasound imaging using a single ultrasound transducer. In a first mode, transducer elements are activated so that ultrasound signals are transmitted from the contact surface at one or more directions normal to the contact surface. In a second mode, a first subset of the transducer elements are activated so that parallel ultrasound signals are transmitted from the contact surface. In a third mode, a second subset of the transducer elements are activated so that ultrasound signals are steered from the second subset of transducer elements.

### CROSS-REFERENCE TO RELATED APPLICATIONS

This application claims the benefit of U.S. Provisional Patent Application No. 62/424,152 entitled “TRANSDUCER ADAPTERS FOR ALLOWING MULTIPLE MODES OF ULTRASOUND IMAGING USING A SINGLE ULTRASOUND TRANSDUCER” filed on Nov. 18, 2016, which is incorporated by reference it its entirety in this disclosure.

### FIELD

The present disclosure relates generally to ultrasound imaging, and particularly, methods and apparatus that enable at least three modes of ultrasound imaging using a single ultrasound transducer.

### BACKGROUND

Traditional ultrasound systems are typically used with a number of different ultrasound probes that are designed to image different parts of the body. These different types of ultrasound probes have different transducer element configurations that make them suitable for imaging different parts of the body.

For example, a phased-array probe typically has a small footprint that allows the probe to be positioned on parts of the body that have constricted space (e.g., in the intercostal space in between a patient's ribs). Since imaging the heart is a common use for this type of probe, it is also called a cardiac probe.

In another example, a sequential curvilinear-array probe (also called a convex or curved probe) contains a larger footprint, with the transducer elements on the probe being positioned on a curve to provide a wide field of view. This configuration makes the curvilinear array probe suitable for imaging the abdomen.

In a further example, a sequential linear array probe may similarly have a wider footprint than that of a phased-array probe. Unlike a cardiac probe or a curvilinear probe, the linear probe directs parallel ultrasound signals from its linear transducer array to provide substantially similar lateral resolution in the near and far field. Linear array probes may be used in various applications, such as vascular.

Using different probes to examine different parts of the body is inconvenient. For example, in examinations performed in an emergency medicine context (e.g., during a Focused Assessment with Sonography in Trauma (FAST) examination), it is desirable to quickly examine multiple internal organs to arrive at a quick medical assessment. The time delay caused by the switching of probes may delay the performance of such examinations.

There is thus a need for improved methods and apparatus for imaging different areas of a patient using the same ultrasound probe.

### BRIEF DESCRIPTION OF THE DRAWINGS

Non-limiting examples of various embodiments of the present disclosure will next be described in relation to the drawings, in which:

FIG. 1 shows different imaging modes of an ultrasound imaging transducer, in accordance with at least one embodiment of the present invention;

FIG. 2 shows the time delays and apertures used to perform beamforming during operation of the ultrasound imaging transducer in a first imaging mode, in accordance with at least one embodiment of the present invention;

FIG. 3 shows the time delays and apertures used to perform beamforming during operation of an ultrasound imaging transducer in a second imaging mode, in accordance with at least one embodiment of the present invention;

FIG. 4 shows the time delays and apertures used to perform beamforming during operation of an ultrasound imaging transducer in a third imaging mode, in accordance with at least one embodiment of the present invention;

FIG. 5 is a flowchart diagram showing steps of a method for generating ultrasound images with an ultrasound imaging transducer, in accordance with at least one embodiment of the present invention; and

FIG. 6 shows a functional block diagram of an ultrasound machine, in accordance with at least one embodiment of the present invention.

### DETAILED DESCRIPTION

In a first broad aspect of the present disclosure, there is provided an ultrasound imaging method, involving: imaging in a first mode using a transducer including a plurality of transducer elements and a contact surface, wherein when imaging in the first mode, the plurality of transducer elements are activated and a first plurality of ultrasound signals are transmitted from the contact surface at one or more directions normal to the contact surface; imaging in a second mode different from the first mode, wherein when imaging in the second mode, a first subset of the plurality of transducer elements are activated and a second plurality of parallel ultrasound signals are transmitted from the contact surface; and imaging in a third mode different from the first mode and the second mode, wherein when imaging in the third mode, a second subset of the plurality of transducer elements are activated and a third plurality of ultrasound signals are steered from the second subset of the plurality of transducer elements.

In some embodiments, the plurality of transducer elements are configured in a curved geometry.

In some embodiments, when imaging in the second mode, a plurality of apertures within the first subset of the plurality of transducer elements are sequentially pulsed.

In some embodiments, when imaging in the second mode, the method further includes steering at least one of the second plurality of parallel ultrasound signals transmitted from the contact surface in a direction away from normal to the contact surface, so that the steered ultrasound signal is parallel with the remaining of the second plurality of parallel ultrasound signals.

In some embodiments, when imaging in the second mode, the second plurality of parallel ultrasound signals form parallel scanlines that generate a substantially rectangular ultrasound image.

In some embodiments, when imaging in the second mode, the first subset of the plurality of transducer elements excludes one or more transducer elements on the periphery of the plurality of transducer elements.

In some embodiments, the imaging in the third mode includes pulsing the second subset of the plurality of transducer elements in a phased manner to generate the third plurality of ultrasound signals.

In some embodiments, when imaging in the third mode, each of the third plurality of ultrasound signals is steered in a respective different direction so that a sector image is generated.

In some embodiments, when imaging in the third mode, a single aperture within the second subset of the plurality of transducer elements is successively pulsed with a plurality of different time delays.

In another broad aspect of the present disclosure, there is provided an ultrasound imaging machine, including: an ultrasound processor; and a transducer communicably coupled to the ultrasound processor, the transducer including a plurality of transducer elements and a contact surface; wherein the ultrasound imaging machine is: operable in a first mode in which the ultrasound processor activates the plurality of transducer elements and a first plurality of ultrasound signals are transmitted from the contact surface at one or more directions normal to the contact surface; operable in a second mode different from the first mode, and in the second mode, the ultrasound processor activates a first subset of the plurality of transducer elements and a second plurality of parallel ultrasound signals are transmitted from the contact surface; and operable in a third mode different from the first mode and the second mode, and in the third mode, the ultrasound processor activates a second subset of the plurality of transducer elements and a third plurality of ultrasound signals are steered from the second subset of the plurality of transducer elements.

In some embodiments, the plurality of transducer elements are configured in a curved geometry.

In some embodiments, when operating in the second mode, a plurality of apertures within the first subset of the plurality of transducer elements are sequentially pulsed.

In some embodiments, when operating in the second mode, the ultrasound processor steers at least one of the second plurality of parallel ultrasound signals transmitted from the contact surface in a direction away from normal to the contact surface, so that the steered ultrasound signal is parallel with the remaining of the second plurality of parallel ultrasound signals.

In some embodiments, when operating in the second mode, the second plurality of parallel ultrasound signals form parallel scanlines that generate a substantially rectangular ultrasound image.

In some embodiments, when operating in the second mode, the first subset of the plurality of transducer elements excludes one or more transducer elements on the periphery of the plurality of transducer elements.

In some embodiments, the operating in the third mode includes pulsing the second subset of the plurality of transducer elements in a phased manner to generate the third plurality of ultrasound signals.

In some embodiments, when operating in the third mode, each of the third plurality of ultrasound signals is steered in a respective different direction so that a sector image is generated.

In some embodiments, when operating in the third mode, a single aperture within the second subset of the plurality of transducer elements is successively pulsed with a plurality of different time delays.

In another broad aspect of the present disclosure, there is provided an ultrasound transducer, capable of being communicably coupled to an ultrasound processor, the ultrasound transducer including: a contact surface; and a plurality of transducer elements positioned proximate to the contact surface, wherein when the ultrasound transducer is communicably coupled to the ultrasound processor, the ultrasound processor is configured to: in a first imaging mode, activate the plurality of transducer elements so that a first plurality of ultrasound signals are transmitted from the contact surface at one or more directions normal to the contact surface; in a second imaging mode different from the first imaging mode, activate a first subset of the plurality of transducer elements, so that a plurality of parallel ultrasound signals are transmitted from the contact surface; and in a third imaging mode different from the first imaging mode and the second imaging mode, activate a second subset of the plurality of transducer elements, so that a third plurality of ultrasound signals are steered from the second subset of the plurality of transducer elements.

In some embodiments, when imaging in the second imaging mode, the ultrasound processor is further configured to steer at least one of the second plurality of parallel ultrasound signals transmitted from the contact surface in a direction away from normal to the contact surface, so that the steered ultrasound signal is parallel with the remaining of the second plurality of parallel ultrasound signals.

For simplicity and clarity of illustration, where considered appropriate, reference numerals may be repeated among the figures to indicate corresponding or analogous elements or steps. In addition, numerous specific details are set forth in order to provide a thorough understanding of the exemplary embodiments described herein. However, it will be understood by those of ordinary skill in the art that the embodiments described herein may be practiced without these specific details. In other instances, certain steps, signals, protocols, software, hardware, networking infrastructure, circuits, structures, techniques, well-known methods, procedures and components have not been described or shown in detail in order not to obscure the embodiments generally described herein.

Furthermore, this description is not to be considered as limiting the scope of the embodiments described herein in any way. It should be understood that the detailed description, while indicating specific embodiments, are given by way of illustration only, since various changes and modifications within the scope of the disclosure will become apparent to those skilled in the art from this detailed description. Accordingly, the specification and drawings are to be regarded in an illustrative, rather than a restrictive, sense.

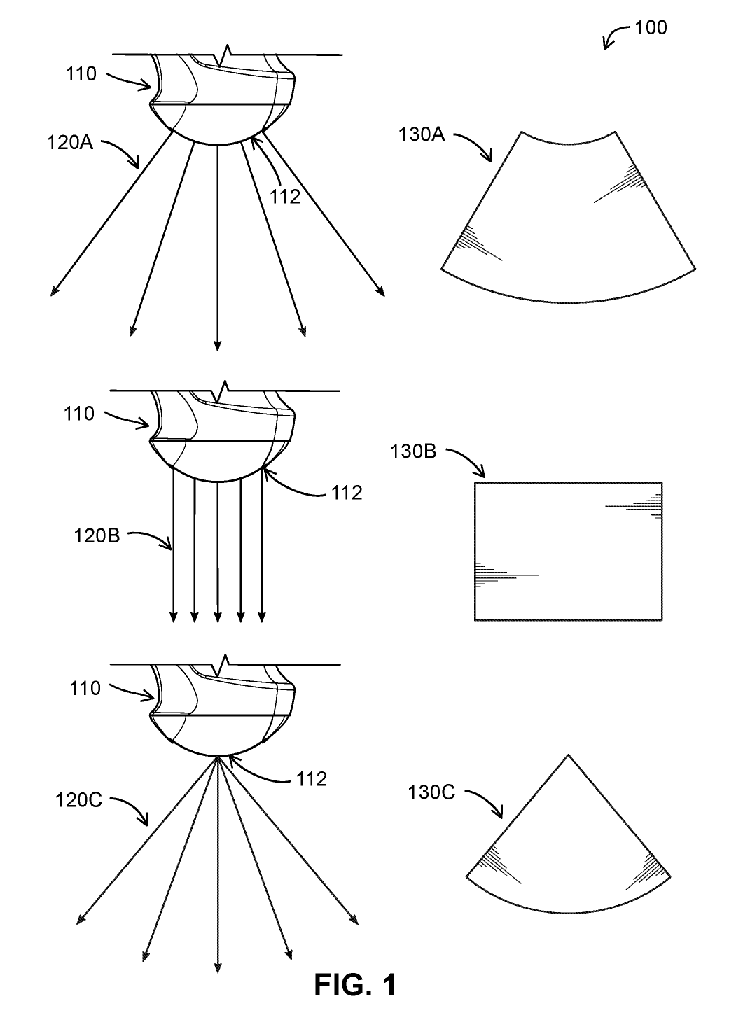

Referring to FIG. 1, shown there generally as 100 are different imaging modes of an ultrasound imaging transducer, in accordance with at least one embodiment of the present invention. As shown, the probe head portion of an ultrasound imaging transducer 110 is viewable. The transducer 110 may have a contact surface 112 that may be placed against the skin of a patient to perform examinations. In the illustrated embodiment, the transducer 110 has a curvilinear or convex footprint. A transducer array with a corresponding curved geometry may be positioned proximate to the contact surface 112 of the transducer 110.

FIG. 1 shows at least three modes of ultrasound imaging that may be performed using a single transducer 110. These at least three different imaging modes may be used to image different parts of a patient and/or generate different types of ultrasound images.

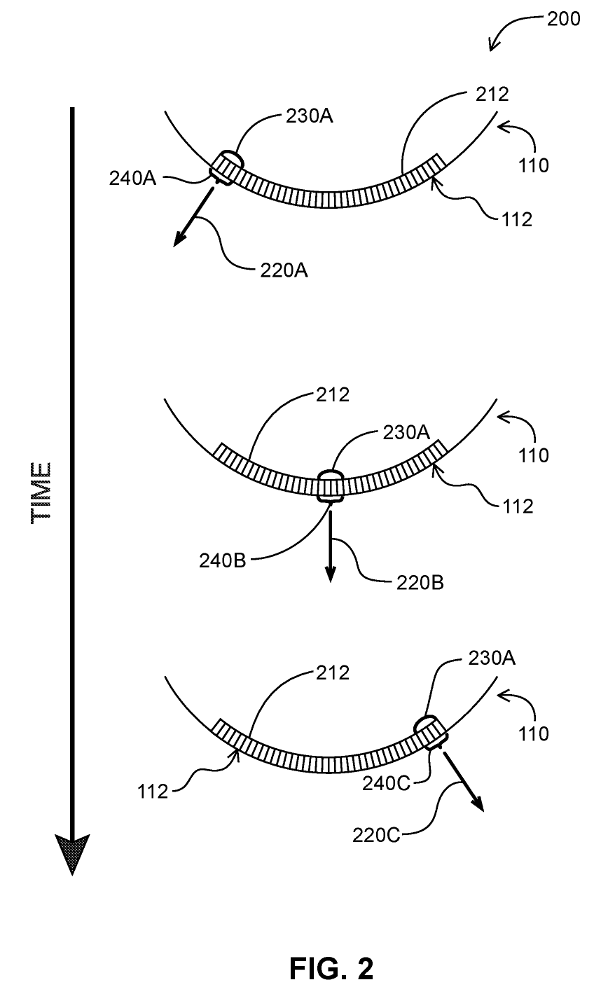

In a first imaging mode, the example curvilinear transducer 110 may be operated in a conventional manner. For example, this may involve activating the transducer elements proximate to the contact surface 112 and transmitting a first plurality of ultrasound signals from the contact surface 112 in one or more directions normal to the contact surface 112. In the illustrated embodiment, the transducer elements are arranged in a curved geometry and the contact surface 112 is curved. As discussed below with respect to FIG. 2, different apertures may be sequentially pulsed across the transducer array. This results in an ultrasound image having a relatively wide field of view. When imaging in the first imaging mode, images that are generated may have the shape 130A that is typical for a curvilinear transducer 110.

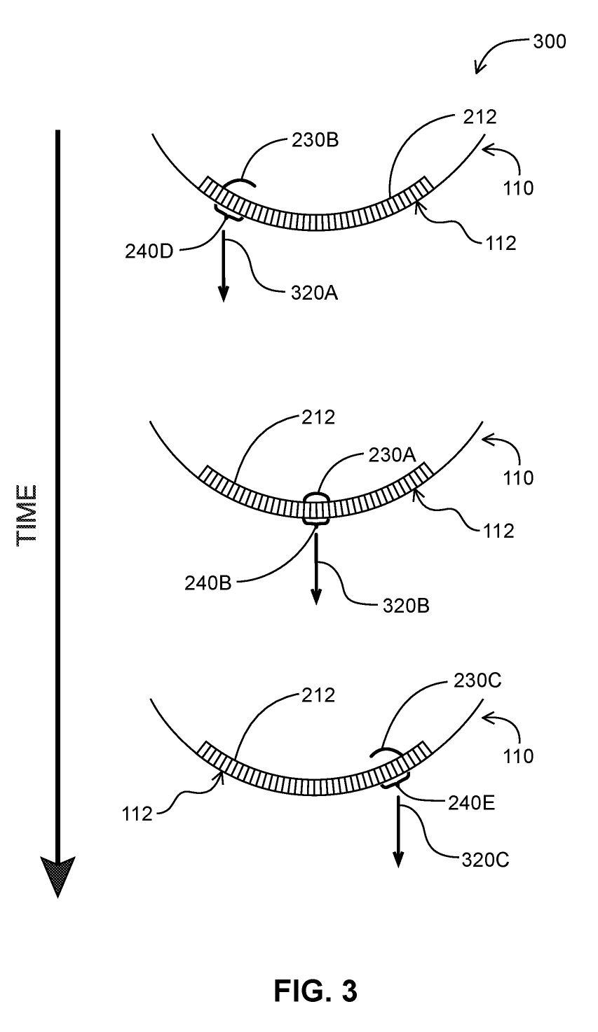

In a second imaging mode, the example curvilinear transducer 110 may be configured to activate only a first subset of the available transducer elements. As discussed below in greater detail with respect to FIG. 3, when imaging in the second mode, a number of apertures within the first subset of the plurality of transducer elements can be sequentially pulsed to generate and transmit a set of parallel ultrasound signals 120B from the contact surface 112. In various embodiments, the set of parallel ultrasound signals 120B may form parallel scanlines that generate a substantially rectangular ultrasound image 130B. The substantially rectangular image 130B may be similar to an ultrasound image conventionally generated by an ultrasound probe having a linear transducer geometry. For example, the rectangular ultrasound image 130B may have a consistent lateral resolution at various imaging depths.

Referring still to FIG. 1, in a third imaging mode, the example sequential curvilinear transducer 110 may be configured to activate a second subset of the available transducer elements. This second subset of transducer elements may be different from the first subset noted above for the second imaging mode. When imaging in the third imaging mode, a set of ultrasound signals can be steered from the second subset of transducer elements. For example, the second subset of transducer elements may be pulsed in a phased manner and steered in a respective different direction so that a fan-shaped (e.g., sector) image 130C is generated. The sector image 130C may be similar to an ultrasound image conventionally generated by an ultrasound probe with a phased array transducer geometry. For example, due to the phased nature of the ultrasound signals being transmitted, lateral resolution may be better in the near field than in the far field of the ultrasound image 130C. Additional teachings related to how subsets of the transducer elements within a transducer array may be activated and selectively steered are discussed in Applicant's U.S. patent application Ser. No. 15/207,203, which is hereby incorporated by reference in its entirety.

Each of the three imaging modes shown in FIG. 1 are traditionally associated with a transducer type. For example, image type 130A is generally associated with a curvilinear probe; image type 130B is generally associated with a linear probe; and image type 130C is generally associated with a phased array probe. However, the present embodiments may allow for at least these three modes of ultrasound imaging to be achieved using a single ultrasound transducer 110 with the same contact surface 112. This may enhance user convenience by removing the need to switch probes. For example, the present embodiments may be desirable in emergency medicine contexts where it is desirable to quickly examine multiple internal organs to arrive at a quick medical assessment.

Referring to FIG. 2, shown there generally as 200 are the time delays and apertures used to perform beamforming during operation of the ultrasound imaging transducer in a first imaging mode, in accordance with at least one embodiment of the present invention. In discussing FIG. 2, reference will also be made to various elements shown in FIG. 1.

As discussed above, the first imaging mode may configure a transducer 110 to operate in manner similar to the conventional operation of a sequential curvilinear-array transducer (e.g., by pulsing transducer elements sequentially across its transducer array). As will be understood by persons skilled in the art, beamforming involves applying a time delay to when adjacent transducer elements 212 are pulsed so that the interference pattern generated by ultrasound signals 120A (as shown in FIG. 1) form a beam when projected. By varying the time delay and sequence in which the transducer elements 212 within a group are pulsed, the beam can be focused so that echo signals resulting from the beam are received as reflections from different tissue structures in a volume of interest.

FIG. 2 shows a simplified view of a transducer head of ultrasound transducer 110 with its constituent transducer elements 212 positioned proximate to the contact surface 112 of the ultrasound transducer 110. FIG. 2 also shows how the transducer elements 212 are pulsed at three example points in time during generation of an ultrasound image in the first imaging mode. To generate an ultrasound image in conventional operation of a sequential transducer 110, ultrasound beams are transmitted from different groups of adjacent transducer elements 212 sequentially and successively across the transducer head. These ultrasound beams result in the formation of scanlines that collectively generate the curvilinear ultrasound image 130A (as shown in FIG. 1). The position(s) of the transducer elements 212 on the transducer head that get pulsed to generate an ultrasound signal may be called the “aperture”. As will be understood by persons skilled in the art, ultrasound operation may involve a transmit aperture and a receive aperture. The transmit aperture refers to the transducer elements 212 that are activated when the ultrasound signals 120A (as shown in FIG. 1) are generated, and the receive aperture refers to the transducer elements 212 that receive echo energy in response. The two apertures may be different such that they include different groups of transducer elements 212. Unless specifically indicated, the term “aperture” refers to the transmit aperture herein.

At the first point in time, the aperture 240A is on the leftmost portion of the transducer head so that a group of adjacent transducer elements 212 there are pulsed. This group of adjacent transducer elements 212 are pulsed according to a time delay 230A. A time delay 230 is illustrated herein as an arc that represents the sequence of activation when the transducers elements 212 are pulsed. As shown, the outermost transducer elements 212 of the aperture 240A are pulsed first, and then transducer elements 212 towards the center of the aperture 240A are progressively pulsed. This type of time delay 230A will generate an ultrasound beam 220A that focuses in a direction normal (e.g., orthogonal) to the contact surface 112 on the transducer head.

At the second point in time, the aperture 240B is in the center portion of the transducer head. Since operation of the transducer in the first mode causes the ultrasound signal to be projected in a direction orthogonal to the contact surface 112 of the transducer head, the same time delay 230A is applied to the aperture 240B to generate the ultrasound beam 220B.

At the third point in time, the aperture 240C is in the rightmost portion of the transducer head. A same time delay 230A is again applied to generate an ultrasound beam 220C that is perpendicular to the contact surface 112 of the transducer head at the position of the aperture 240C. Over time, various scanlines can be used to collectively form a curvilinear image type 130A (as shown in FIG. 1).

Referring to FIG. 3, shown there generally as 300 are the time delays and apertures used to perform beamforming during operation of an ultrasound imaging transducer in a second imaging mode, in accordance with at least one embodiment of the present invention. In discussing FIG. 2, reference will also be made to various elements shown in FIG. 1.

As noted above, when imaging in the second mode, the ultrasound transducer may transmit parallel ultrasound signals 120B (as shown in FIG. 1) similar to what may traditionally be emitted from a traditional linear-array transducer. In some embodiments where the ultrasound transducer 110 has its transducer array arranged in a curved geometry, some of the ultrasound signals 120B may be steered in a direction away from normal to the contact surface 112, so that the steered ultrasound signal is parallel with the remaining of the second plurality of parallel ultrasound signals. This may be achieved by altering the time delays and sequence in which the elements 212 of the transducer array in the example curvilinear transducer 110 are pulsed. For example, varying the time delay and sequence in which the transducer elements 212 within a subset of the transducer elements are pulsed, the beams can be steered so as to provide ultrasound beams that are emitted from the contact surface 112 that mimic those typically emitted from a linear-array probe.

Like FIG. 2, FIG. 3 shows a simplified view of a transducer head of ultrasound transducer 110 with its constituent transducer elements 212 positioned proximate to the contact surface 112 of the ultrasound transducer 110. FIG. 2 also shows how the transducer elements 212 are pulsed at three example points in time during generation of an ultrasound image in the second imaging mode. To generate a substantially rectangular image, ultrasound beams are transmitted from selected groups of adjacent transducer elements 212 sequentially and successively across a portion of the transducer array that excludes the peripheral transducer elements. These ultrasound beams result in the formation of parallel scanlines that collectively generate the substantially rectangular ultrasound image 130B (as shown in FIG. 1).

At the first point in time, the aperture 240D is on a left portion of the transducer array, so that a group of adjacent transducer elements 212 there are pulsed. This group of adjacent transducer elements 212 are pulsed according to a time delay 230B. The time delay 230B is illustrated as an arc that represents the sequence of activation when the transducers elements 212 are pulsed. As shown, the time delay 230B shown has the leftmost transducer elements 212 within the aperture 240D being activated first and then progressively shifting to the right of the aperture 240D in the sequence and manner represented by the time delay 230B. The time delay 230B will cause the ultrasound signal 320A to be steered in a manner that is angled away from the azimuth/normal at aperture 240D.

At the second point in time, the aperture 240B is in the center portion of the transducer array. Like the time delay 230A shown in FIG. 2, the time delay 230A that is applied at this second point in time starts with the outermost transducer elements 212 of the aperture 230A being pulsed first, and then transducer elements 212 towards the center of the aperture 240B are progressively pulsed. This type of time delay 230A may generate an ultrasound beam 320B that is unsteered, and focuses in a direction normal/orthogonal to the contact surface 112 of the probe head at the aperture 240B. This is because at the second point in time in FIG. 3, the ultrasound signal 320B desired to be projected happens to be normal/orthogonal to the contact surface 112 of the transducer head.

At the third point in time, the aperture 240E is on a right portion of the transducer array. This group of adjacent transducer elements 212 are pulsed according to a time delay 230C. As shown, the time delay 230C has the rightmost transducer elements 212 within the aperture 240E being activated first and then progressively shifting to the left of the aperture 240E in the sequence and manner represented by the time delay 230C. The time delay 230C may cause the ultrasound signal 320C to be angled away from the azimuth/normal at aperture 240E.

Collectively, the various ultrasound signals 320A, 320B, 320C are configured so that they are parallel with each other. This may allow a substantially rectangular image 130B (as shown in FIG. 1) to be generated, in a manner similar to that which would be generated from a traditional linear-array probe.

Traditional linear-array probes have a generally planar contact surface area. However, in the example embodiment illustrated in FIG. 3, the transducer 110 is provided with a transducer array arranged in a curved geometry having a curved contact surface 112. Because of this curvature, it is possible that the signals received on the outer edges of the subset of transducer elements 212 used for imaging have a different depth-origin point (e.g., zero point) than those in the middle of the subset of the transducer elements 212. To accurately reflect this, in some embodiments, the top edge of the image generated in the second imaging mode might have a slight curvature that reflects the curved geometry of the transducer elements 212 used to acquire the images. Additionally or alternatively, if a uniform top edge of the rectangular image is desired, the depth-origin of the image may be set to the lowest point of the curvature of the subset of transducer elements 212 used to perform imaging (e.g., in the middle of the transducer array); and suitable adjustments may be made when displaying the imaging depth of the scanlines acquired from any aperture that is higher than the lowest point due to the curvature. For example, these adjustments may include ignoring any echo data acquired for depths less than the lowest point, and only begin displaying image data at depths starting from the lowest point. In this way, the images 130B (as shown in FIG. 1) may not be uniformly rectangular in all instances, but instead, may be substantially rectangular.

To generate a rectangular ultrasound image using the full width of the available transducer elements, it may be necessary to exert an overly forceful application of the curved transducer head against the tissue being imaged. While this may allow the transducer elements 212 on the periphery of the transducer array to have sufficient contact and coupling to the skin, this may cause discomfort for the patient being imaged and/or be unergonomic for the ultrasound operator.

Instead of using the full width of available transducer elements to perform imaging in the second mode, in some embodiments, only a subset of all the available transducer elements 212 may be used. For example, as shown in FIG. 3, the subset of transducer elements 212 activated when imaging in the second mode excludes transducer elements 212 on the outer edges (e.g., periphery) of the transducer elements 212 in the transducer array. While using a subset of transducer elements 212 in this manner may result in a narrower width for the resultant substantially rectangular image 130B, it may also allow imaging to be performed in the second mode without requiring undue forceful application of the curved probe head against the tissue being imaged (or any associated compression of the tissue, for example). In this embodiment, since the periphery transducer elements 212 are not being activated for the purpose of imaging, they do not need to have contact with the skin. As a result, simply resting the ultrasound transducer 110 on the skin of the tissue being imaged may provide sufficient contact and coupling for the subset of transducer elements 212 being activated to image in the second mode. This may reduce patient discomfort and/or improve ergonomics for the ultrasound operator. Depending on the nature of the imaging desired to be performed, the subset of the transducer elements 212 selected to be activated during the second imaging mode may be wider or narrower in various embodiments.

When operating in the second imaging mode, the frequency of the ultrasound signals 120B (as shown in FIG. 1) emitted may be lower than what is typically transmitted from a traditional linear ultrasound probe. In addition, the transducer elements 212 used in the example curvilinear probe 110 may have a coarser elevation (also called slice thickness) resolution. Notwithstanding, the lower frequency and thicker slice thickness may still be suitable for certain types of medical examinations (e.g., vascular)—especially if consistent lateral resolution in the near and far field is desirable. The second imaging mode may also be suitable if speed of examination is desirable and it is preferred to switch imaging modes rather than use a dedicated linear-array ultrasound probe.

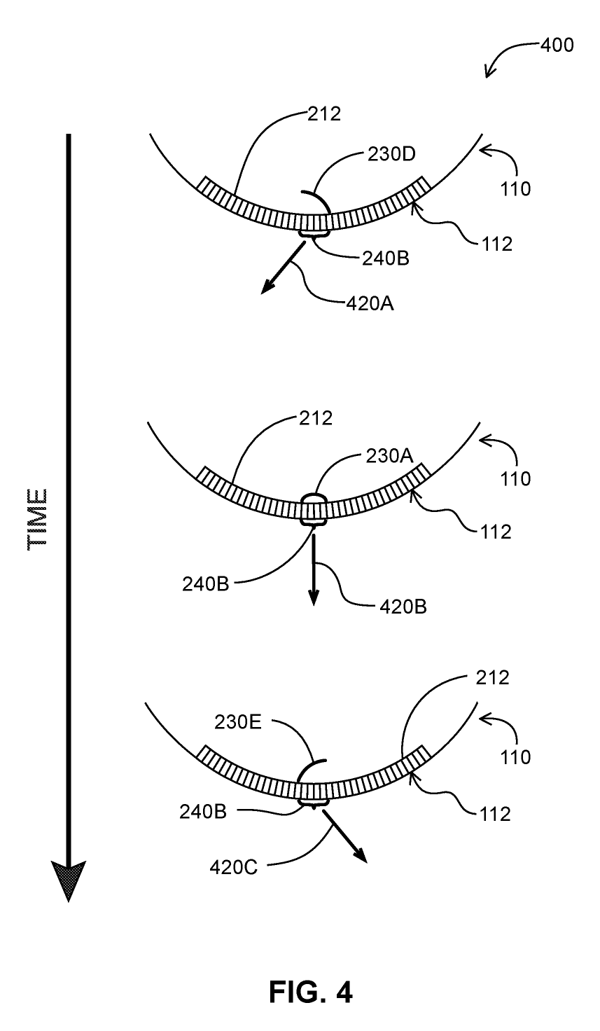

Referring to FIG. 4, shown there generally as 400 are the time delays and apertures used to perform beamforming during operation of an ultrasound imaging transducer in a third imaging mode, in accordance with at least one embodiment of the present invention. In discussing FIG. 4, reference will also be made to various elements shown in FIG. 1.

In some embodiments, when imaging in the third mode, a different subset of the transducer elements 212 (different from the subset used in the second imaging mode) may be successively pulsed with different time delays. In some embodiments, this subset may form a single aperture from which ultrasound signals 120C (as shown in FIG. 1) may be steered in multiple directions.

Like FIGS. 2 and 3, FIG. 4 shows a simplified view of a transducer head of ultrasound transducer 110 with its constituent transducer elements 212 positioned proximate to the contact surface 112 of the ultrasound transducer 110. FIG. 2 also shows how the transducer elements 212 are pulsed at three example points in time during generation of an ultrasound image in the third imaging mode.

At the first point in time, a time delay 230D can be applied to an aperture 240B on the transducer head. Referring simultaneously to FIGS. 2 and 3, it can be seen that the shape of the time delay 230D applied is different from the time delay 230A repeatedly applied in FIG. 2, and also different from the time delays 230B, 230C for steering ultrasound signals in FIG. 3. As compared to the time delay 230A used in FIG. 2, the difference in time delay being applied to the aperture 240B causes the resultant ultrasound signal 420A to be steered in a direction that is different from normal/orthogonal to the contact surface 112 of the transducer head at the point of the aperture 240B. Specifically, the particular time delay 230D shown has the rightmost transducer elements 212 within the aperture 240B being activated first and then progressively shifting to the left of the aperture 240B in the sequence and manner represented by the time delay 230D. The time delay 230D may cause the ultrasound signal 420A to be directed in a direction to the left of normal to the contact surface 112 at the point of the aperture 240B.

At the second point in time, a time delay 230A is applied to the same aperture 240B that was activated during the first point in time. As can be seen, this time delay is different from the time delay 230D applied during the first point in time. Referring simultaneously to FIG. 2, it can be seen that the time delay 230A applied at the second point in time in FIG. 4 is substantially similar to the time delay 230A applied at various points in time in FIG. 2 to various apertures 240A, 240B, 240C. This is because at the second point in time in FIG. 4, the ultrasound signal 420B desired to be projected happens to be normal/orthogonal to the contact surface 112 of the transducer head.

At the third point in time, a time delay 230E is applied again to the same aperture 240B that was activated during the first and second points in time. The time delay 230E is different from the time delays 230D, 230A applied at the first and second points in time. As shown, the time delay 230E applied is in the reverse sequence and timing to the time delay 230D applied at the first point in time of FIG. 4. This results in the ultrasound signal 420C generated being directed to the right at the point of the aperture 240B.

Referring simultaneously to FIGS. 2-4, it can be seen that when operating in the first mode (FIG. 2), the ultrasound transducer 110 pulses different apertures 240A, 240B, 240C along the transducer head with the same time delay 230A so as to cause ultrasound signals 220A, 220B 220C to be projected in respective directions that are normal/orthogonal to the contact surface 112 of the transducer head at the locations of each aperture 240A, 240B, 240C. In the second mode (FIG. 3), the ultrasound transducer 110 pulses different apertures 240D, 240B, 240E within a subset of all the available transducer elements 212 using different time delays so as to direct (and steer, as necessary) the ultrasound signals 320A, 320B, 320C in parallel directions. In the third mode (FIG. 4), the ultrasound transducer 110 repeatedly pulses a single aperture 240B on the transducer head but with different time delays 230D, 230A, 230E to steer the respective ultrasound signals 420A, 420B, 420C in multiple directions.

In this manner, a single ultrasound transducer 110 may be operable in three different imaging modes: a first conventional imaging mode; a second “virtual linear” mode; and a third “virtual phased-array” mode. These three modes may mimic the operation of three separate ultrasound transducers without requiring the purchase of multiple probes or switching of probes during examination.

Although FIGS. 3-4 described herein have been shown and discussed with respect to activating example subsets of available transducer elements 212 in the second mode and third mode, different selections of transducer element 212 subsets may be possible. For example, in in an example embodiment, the subset of transducer elements 212 activated in the second “virtual linear” imaging mode may include up to two-thirds (⅔^{rd}) of all available transducer elements 212 on the ultrasound transducer 110. In another example embodiment, the subset of the transducer elements activated in the third “virtual phased-array” imaging mode may include up to one-third (⅓^{rd}) of all available transducer elements 212 on the ultrasound transducer 110. In various embodiments, the size and location of apertures, as well as time delays used to steer and direct ultrasound signals may also be different from what is illustrated in the figures herein. Additionally or alternatively, one or more of time delay, sequence, steering angle, transmit aperture size, transmit aperture location, receive aperture size, receive aperture location, and/or image zero point may be modified to suit desired imaging qualities in any of the imaging modes.

Moreover, while the transducer 110 shown herein is illustrated with a curved transducer geometry, different transducer geometries may be possible. For example, in some embodiments, there may be different curvatures of transducer geometry with fewer or more transducer elements 212. Additionally or alternatively, in some embodiments, the transducer geometry of the transducer 110 with which the present embodiments may be practiced may be linear.

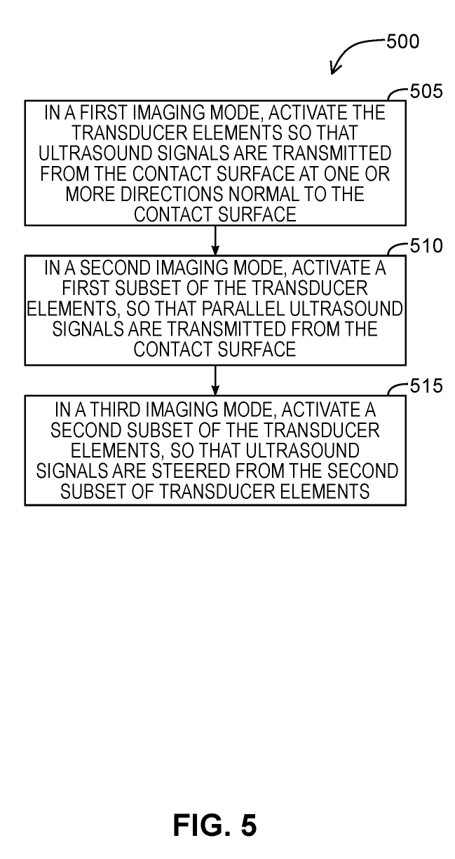

Referring to FIG. 5, shown there generally as 500 is a flowchart diagram showing steps of a method for generating ultrasound images with an ultrasound imaging transducer, in accordance with at least one embodiment of the present invention. In some embodiments, the present disclosure may be considered methods of performing ultrasound imaging that allows for switching from amongst at least three imaging modes using a single ultrasound transducer 110. In discussing the method of FIG. 5, reference will also be made to FIG. 1. For example, the method of FIG. 5 may be performed by the ultrasound transducer 110 shown in FIG. 1.

At 505, in a first imaging mode, the ultrasound transducer 110 may activate the transducer elements 212 (as shown in FIGS. 2-4) so that ultrasound signals 120A (as shown in FIG. 1) may be transmitted from the contact surface 112 at one or more directions normal to the contact surface 112. Using the example time delays and apertures discussed above with respect to FIG. 2, a curvilinear ultrasound image 130A may be generated.

At 510, in a second imaging mode, the ultrasound transducer 110 may activate a first subset of the available transducer elements 212 (as shown in FIGS. 2-4), so that parallel ultrasound signals 120B (as shown in FIG. 1) may be transmitted from the contact surface 112. Using the example time delays and apertures discussed above with respect to FIG. 3, parallel scanlines may be transmitted and a substantially rectangular ultrasound image 130B may be generated from the associated echoes.

At 515, in a third imaging mode, the ultrasound transducer 110 may activate a second subset of the transducer elements 212 (as shown in FIG. 2-4), so that ultrasound signals 120C (as shown in FIG. 1) may be steered from the second subset of the plurality of transducer elements 212. Using the example time delays and apertures discussed above with respect to FIG. 4, ultrasound signals may be steered in a phased manner in different respective directions to generate a sector image 130C.

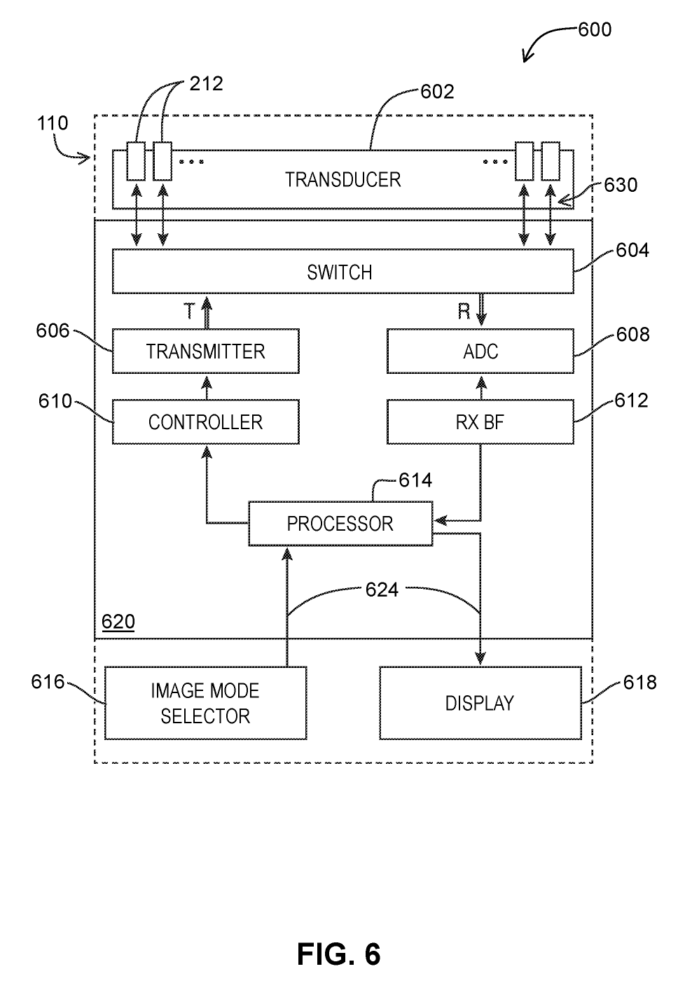

Referring to FIG. 6, shown there generally as 600 is a functional block diagram of an ultrasound machine, in accordance with at least one embodiment of the present invention. The ultrasound machine 600 may include a transducer 110 that may form part of ultrasound machine 600. The transducer 110 may be have a transducer array 602 with constituent transducer elements 212. The transducer array 602 may be positioned proximate to a contact surface 112 (not shown in FIG. 6) that is placed against a surface (e.g., skin) covering a volume to be imaged.

A transmitter 606 may be provided to energize the transducer elements 212 to produce the ultrasound signals discussed above. Another group of transducer elements 212 may then form the receive aperture to convert the received ultrasound energy into analog electrical signals which may then be sent through a set of transmit/receive (T/R) switches 604 to a number of channels of echo data. A set of analog-to-digital converters (ADCs) 608 nay digitise the analog signals from the switches 604. The digitised signals may then be sent to a receive beamformer 612.

Transmitter 606 and receive beamformer 612 may be operated under the control of a scan controller 610. Receive beamformer 612 may combine the separate echo signals from each channel using pre-calculated time delay and weight values that may be stored in a coefficient memory (not shown) to yield a single echo signal which represents the received energy from a particular scanline. Under the direction of the scan controller 610, the ultrasound machine 600 may generate and process additional transmit and receive events to produce the multiple scanlines required to form an ultrasound image. Ultrasound images are typically made up of 50 to a few hundred lines. Typically, the number of scanlines of an ultrasound image generated from a sequential transducer may correspond to the number of transducer elements 212 in the transducer array 602.

However, when the transducer 110 described herein is operated in the second or third mode, the scanlines generated from the respective subsets of the transducer elements 212 may not correlate to the number of available transducer elements 212 present in the transducer array 602. Instead, the number of scanlines may correspond to the size of the subset selected for a given mode (e.g., for the second or “virtual linear” imaging mode, the desired line density selected for a substantially rectangular image); or the configured angular separation of the transmitted ultrasound signals that generate echo signals which form the sector image (e.g., for the third or “virtual phased array” imaging mode).

In some embodiments, the apparatus and methods described herein may be employed using both Single Line Acquisition (SLA) and Multi-Line Acquisition (MLA) techniques. As will be understood by persons skilled in the art, images generated using SLA techniques have a single receive scanline for a single transmitted ultrasound signal and images generated using MLA techniques have multiple receive scanlines for a single transmitted ultrasound signal. This may allow ultrasound systems that employ MLA techniques to have improved frame rates. In further embodiments, synthetic aperture techniques may be used to improve lateral resolution of an ultrasound image.

An ultrasound processor 614 may be in communication with the receive beamformer 612 and may apply the necessary processing steps to combine multiple scanlines from these different transmit events to yield image data. The processor 614 may communicate this image data via a data link 624 to a display device 618. Data link 624 may include a cable, a wireless connection, or the like. Display device 618 may display generated ultrasound images. In some embodiments, the display device 618 may not be separate, and instead be provided as an integrated part of the ultrasound machine 600. In the latter case, the data link 624 may be a data bus or other suitable connector between the processor 614 and the display 618.

The image mode selector 616 may receive input to select between the first, second, and third imaging modes discussed herein. The image mode selector 616 may be provided in the form of any physical or software-based user interface control. For example, in some embodiments, a user control such as a push button, a graphical user interface control, or the like may be operated by an ultrasound operator. The data input selecting the mode of operation may be provided to ultrasound processor 614 via data link 624. In turn, the ultrasound processor 614 may provide a configuration signal to controller 610 to modify the operation of the transmitter 606 and receive beamformer 612 to activate the transducer array 602 in accordance with the selected imaging mode.

In some embodiments, the image mode selector 616 may be provided in a form that links the imaging mode to predetermined pre-sets for imaging certain anatomy or a medical specialty. For example, an ‘Abdomen’ pre-set may be linked to the conventional first curvilinear imaging mode; a ‘Vascular’ pre-set may be linked to the second “virtual linear” imaging mode; and a ‘Cardiac’ pre-set may be linked to the third “virtual phased array” imaging mode.

In some embodiments, the operation of the image mode selector 616 may be performed automatically via suitable software instructions. For example, the processor 614 may be provided with software instructions to automatically detect anatomy present in the ultrasound images being generated, so as to change to the appropriate imaging mode automatically. For example, using neural networks or deep learning algorithms that segment ultrasound images to identify known anatomy, the processor 614 may be configured to switch the imaging mode from one mode to another (e.g., if a beating heart valve is detected in the field of view in the first imaging mode, the processor 614 may be configured to automatically switch to the third “virtual phased-array” cardiac imaging mode).

The embodiments described herein may be used with ultrasound machines 600 having a variety of different form factors. As illustrated in FIG. 6, the transducer head (holding the transducer array 602 with constituent transducer elements 212) is shown in dotted outline in relation to the processing components 620 of the ultrasound machine 600 to illustrate that it can be coupled thereto via any type of communication link 630. For example, in some embodiments, a transducer 110 may just encompass the transducer head, and such transducer 110 may be detachably coupled to the body of the ultrasound machine 600 via a cable or other suitable wired connection. In some such embodiments, the ultrasound machine 600 may include both the processing components 620 and the display 618 and image mode selector 616 in a unitary body.

In certain embodiments, the transducer head and processing components 620 may be provided in a single device (e.g., having a unitary body). In such case, the processor 614 may communicate to display 618 and image mode selector 616 via a wireless communication link. The image mode selector 616 and display 618 is shown in dotted outline to show that they may not form part of the processing components 620 in such embodiments. In some such embodiments, the single device containing the transducer head and processing components 620 may be provided as a wired or wireless handheld probe that is configured to communicate with an external computing device containing a display 618 and is able to provide functionality for the image mode selector 616. In some embodiments, such handheld probe may be provided in a form factor that has a mass that is less than 4.5 kilograms.

Configuring a single transducer head to operate in multiple imaging modes as described herein may be desirable in embodiments where the transducer head and the processing components 620 are provided in a unitary body because it is not possible to remove the transducer head from the body containing the processing components 620. Put another way, configuring the single, non-detachable transducer head to operate in multiple imaging modes may provide enhanced utility of a handheld ultrasound probe.

The various embodiments discussed herein may facilitate imaging multiple patient areas using a single ultrasound transducer 110. For example, when used in a conventional context, a curvilinear probe may be used to image the abdomen. However, with the additional imaging modes discussed herein, the same curvilinear probe may also be used to perform imaging that would typically require two additional probes (e.g., a traditional phased-array cardiac probe and a traditional linear probe). The present embodiments may thus allow the single curvilinear probe to serve the needs that would typically be served by three different ultrasound probes.

While a number of exemplary aspects and embodiments have been discussed above, those of skill in the art will recognize that may be certain modifications, permutations, additions and sub-combinations thereof. While the above description contains many details of example embodiments, these should not be construed as essential limitations on the scope of any embodiment. Many other ramifications and variations are possible within the teachings of the various embodiments.

### INTERPRETATION OF TERMS

Unless the context clearly requires otherwise, throughout the description and the claims:
“comprise”, “comprising”, and the like are to be construed in an inclusive sense, as opposed to an exclusive or exhaustive sense; that is to say, in the sense of “including, but not limited to”;
“connected”, “coupled”, or any variant thereof, means any connection or coupling, either direct or indirect, between two or more elements; the coupling or connection between the elements can be physical, logical, or a combination thereof;
“herein”, “above”, “below”, and words of similar import, when used to describe this specification, shall refer to this specification as a whole, and not to any particular portions of this specification;
“or”, in reference to a list of two or more items, covers all of the following interpretations of the word: any of the items in the list, all of the items in the list, and any combination of the items in the list;
the singular forms “a”, “an”, and “the” also include the meaning of any appropriate plural forms.

Unless the context clearly requires otherwise, throughout the description and the claims:

Words that indicate directions such as “vertical”, “transverse”, “horizontal”, “upward”, “downward”, “forward”, “backward”, “inward”, “outward”, “vertical”, “transverse”, “left”, “right”, “front”, “back”, “top”, “bottom”, “below”, “above”, “under”, and the like, used in this description and any accompanying claims (where present), depend on the specific orientation of the apparatus described and illustrated. The subject matter described herein may assume various alternative orientations. Accordingly, these directional terms are not strictly defined and should not be interpreted narrowly.

Embodiments of the invention may be implemented using specifically designed hardware, configurable hardware, programmable data processors configured by the provision of software (which may optionally comprise “firmware”) capable of executing on the data processors, special purpose computers or data processors that are specifically programmed, configured, or constructed to perform one or more steps in a method as explained in detail herein and/or combinations of two or more of these. Examples of specifically designed hardware are: logic circuits, application-specific integrated circuits (“ASICs”), large scale integrated circuits (“LSIs”), very large scale integrated circuits (“VLSIs”), and the like. Examples of configurable hardware are: one or more programmable logic devices such as programmable array logic (“PALs”), programmable logic arrays (“PLAs”), and field programmable gate arrays (“FPGAs”). Examples of programmable data processors are: microprocessors, digital signal processors (“DSPs”), embedded processors, graphics processors, math co-processors, general purpose computers, server computers, cloud computers, mainframe computers, computer workstations, and the like. For example, one or more data processors in a control circuit for a device may implement methods as described herein by executing software instructions in a program memory accessible to the processors.

For example, while processes or blocks are presented in a given order herein, alternative examples may perform routines having steps, or employ systems having blocks, in a different order, and some processes or blocks may be deleted, moved, added, subdivided, combined, and/or modified to provide alternative or subcombinations. Each of these processes or blocks may be implemented in a variety of different ways. Also, while processes or blocks are at times shown as being performed in series, these processes or blocks may instead be performed in parallel, or may be performed at different times.

The invention may also be provided in the form of a program product. The program product may comprise any non-transitory medium which carries a set of computer-readable instructions which, when executed by a data processor (e.g., in a controller and/or ultrasound processor in an ultrasound machine), cause the data processor to execute a method of the invention. Program products according to the invention may be in any of a wide variety of forms. The program product may comprise, for example, non-transitory media such as magnetic data storage media including floppy diskettes, hard disk drives, optical data storage media including CD ROMs, DVDs, electronic data storage media including ROMs, flash RAM, EPROMs, hardwired or preprogrammed chips (e.g., EEPROM semiconductor chips), nanotechnology memory, or the like. The computer-readable signals on the program product may optionally be compressed or encrypted.

Where a component (e.g. a software module, processor, assembly, device, circuit, etc.) is referred to above, unless otherwise indicated, reference to that component (including a reference to a “means”) should be interpreted as including as equivalents of that component any component which performs the function of the described component (i.e., that is functionally equivalent), including components which are not structurally equivalent to the disclosed structure which performs the function in the illustrated exemplary embodiments of the invention.

Specific examples of systems, methods and apparatus have been described herein for purposes of illustration. These are only examples. The technology provided herein can be applied to systems other than the example systems described above. Many alterations, modifications, additions, omissions, and permutations are possible within the practice of this invention. This invention includes variations on described embodiments that would be apparent to the skilled addressee, including variations obtained by: replacing features, elements and/or acts with equivalent features, elements and/or acts; mixing and matching of features, elements and/or acts from different embodiments; combining features, elements and/or acts from embodiments as described herein with features, elements and/or acts of other technology; and/or omitting combining features, elements and/or acts from described embodiments.

It is therefore intended that the following appended claims and claims hereafter introduced are interpreted to include all such modifications, permutations, additions, omissions, and sub-combinations as may reasonably be inferred. The scope of the claims should not be limited by the preferred embodiments set forth in the examples, but should be given the broadest interpretation consistent with the description as a whole.

What is claimed is:

1. An ultrasound imaging method, comprising:
imaging in a first mode using a transducer comprising a plurality of transducer elements and a contact surface, wherein when imaging in the first mode, the plurality of transducer elements are activated and a first plurality of ultrasound signals are transmitted from the contact surface at one or more directions normal to the contact surface;
imaging in a second mode different from the first mode, wherein when imaging in the second mode, a first subset of the plurality of transducer elements are activated and a second plurality of parallel ultrasound signals are transmitted from the contact surface; and
imaging in a third mode different from the first mode and the second mode, wherein when imaging in the third mode, a second subset of the plurality of transducer elements are activated and a third plurality of ultrasound signals are steered from the second subset of the plurality of transducer elements.

2. The method of claim 1, wherein the plurality of transducer elements are configured in a curved geometry.

3. The method of claim 1, wherein when imaging in the second mode, a plurality of apertures within the first subset of the plurality of transducer elements are sequentially pulsed.

4. The method of claim 3, wherein when imaging in the second mode, the method further comprises steering at least one of the second plurality of parallel ultrasound signals transmitted from the contact surface in a direction away from normal to the contact surface, so that the steered ultrasound signal is parallel with the remaining of the second plurality of parallel ultrasound signals.

5. The method of claim 1, wherein when imaging in the second mode, the second plurality of parallel ultrasound signals form parallel scanlines that generate a substantially rectangular ultrasound image.

6. The method of claim 1, wherein when imaging in the second mode, the first subset of the plurality of transducer elements excludes one or more transducer elements on the periphery of the plurality of transducer elements.

7. The method of claim 1, wherein the imaging in the third mode comprises pulsing the second subset of the plurality of transducer elements in a phased manner to generate the third plurality of ultrasound signals.

8. The method of claim 1, when imaging in the third mode, each of the third plurality of ultrasound signals is steered in a respective different direction so that a sector image is generated.

9. The method of claim 1, wherein when imaging in the third mode, a single aperture within the second subset of the plurality of transducer elements is successively pulsed with a plurality of different time delays.

10. An ultrasound imaging machine, comprising:
an ultrasound processor; and
a transducer communicably coupled to the ultrasound processor, the transducer comprising a plurality of transducer elements and a contact surface;
wherein the ultrasound imaging machine is:
operable in a first mode in which the ultrasound processor activates the plurality of transducer elements and a first plurality of ultrasound signals are transmitted from the contact surface at one or more directions normal to the contact surface;
operable in a second mode different from the first mode, and in the second mode, the ultrasound processor activates a first subset of the plurality of transducer elements and a second plurality of parallel ultrasound signals are transmitted from the contact surface; and
operable in a third mode different from the first mode and the second mode, and in the third mode, the ultrasound processor activates a second subset of the plurality of transducer elements and a third plurality of ultrasound signals are steered from the second subset of the plurality of transducer elements.

11. The ultrasound imaging machine of claim 10, wherein the plurality of transducer elements are configured in a curved geometry.

12. The ultrasound imaging machine of claim 10, wherein when operating in the second mode, a plurality of apertures within the first subset of the plurality of transducer elements are sequentially pulsed.

13. The ultrasound imaging machine of claim 12, wherein when operating in the second mode, the ultrasound processor steers at least one of the second plurality of parallel ultrasound signals transmitted from the contact surface in a direction away from normal to the contact surface, so that the steered ultrasound signal is parallel with the remaining of the second plurality of parallel ultrasound signals.

14. The ultrasound imaging machine of claim 10, wherein when operating in the second mode, the second plurality of parallel ultrasound signals form parallel scanlines that generate a substantially rectangular ultrasound image.

15. The ultrasound imaging machine of claim 10, wherein when operating in the second mode, the first subset of the plurality of transducer elements excludes one or more transducer elements on the periphery of the plurality of transducer elements.

16. The ultrasound imaging machine of claim 10, wherein the operating in the third mode comprises pulsing the second subset of the plurality of transducer elements in a phased manner to generate the third plurality of ultrasound signals.

17. The ultrasound imaging machine of claim 10, when operating in the third mode, each of the third plurality of ultrasound signals is steered in a respective different direction so that a sector image is generated.

18. The ultrasound imaging machine of claim 10, wherein when operating in the third mode, a single aperture within the second subset of the plurality of transducer elements is successively pulsed with a plurality of different time delays.

19. An ultrasound transducer, capable of being communicably coupled to an ultrasound processor, the ultrasound transducer comprising:
a contact surface; and
a plurality of transducer elements positioned proximate to the contact surface, wherein when the ultrasound transducer is communicably coupled to the ultrasound processor, the ultrasound processor is configured to:
in a first imaging mode, activate the plurality of transducer elements so that a first plurality of ultrasound signals are transmitted from the contact surface at one or more directions normal to the contact surface;
in a second imaging mode different from the first imaging mode, activate a first subset of the plurality of transducer elements, so that a plurality of parallel ultrasound signals are transmitted from the contact surface; and
in a third imaging mode different from the first imaging mode and the second imaging mode, activate a second subset of the plurality of transducer elements, so that a third plurality of ultrasound signals are steered from the second subset of the plurality of transducer elements.

20. The ultrasound transducer of claim 19, wherein when imaging in the second imaging mode, the ultrasound processor is further configured to steer at least one of the second plurality of parallel ultrasound signals transmitted from the contact surface in a direction away from normal to the contact surface, so that the steered ultrasound signal is parallel with the remaining of the second plurality of parallel ultrasound signals.
